(19) United States
(12) Patent Application Publication
(10) Pub. No.: US 2003/0003456 A1
(43) Pub. Date: Jan. 2, 2003

(54) Method and system of identifying biologically active molecules

(76) Inventors: Frank Schmitt, Muenchen (DE); Bernhard Schirm, Gauting (DE); Bernd Kramer, Aachen (DE); Knut Baumann, Wuerzburg (DE); Daniel Vitt, Germering (DE)

Correspondence Address: Davidson, Davidson & Kappel LLC, 14th Floor, 485 Seventh Avenue, New York, NY, 10018, US

(21) Appl. No.: 09/885,517

(22) Filed: Jun. 20, 2001

(51) Int. Cl. C12Q 1/68; G06G 7/48; G06G 7/58; G06F 19/00; G01N 33/48; G01N 33/50

(52) U.S. Cl. 435/6; 703/11; 702/19

(57) ABSTRACT

The present invention relates to a method and a system of identifying biologically active molecules. Evaluating receptor or target suitability of molecules is an important task in pharmaceutical drug research. With the increasing employment of automation techniques over the last years within Drug Discovery processes, methods like High-Throughput-Screening (HTS) and High-Throughput-Synthesis have become industry standards in pharmaceutical research. Nowadays, it is possible to test more than 20,000 molecules per day for their biological activities in certain disease targets. Also in the area of chemical synthesis, combinatorial chemistry in combination with automation processes, hundreds of molecules per day can be made physically available. Since based on today's chemical knowledge, more than 10¹⁰⁰ molecules could theoretically be synthesized and tested and several hundreds of thousands molecules are commercially available, computer assisted methods have been developed to select subsets of molecules which are actually supposed to be tested based on their predicted potential of biological activity for certain disease targets.

The present invention relates to a method and a system of identifying biologically active molecules.

Evaluating receptor or target suitability of molecules is an important task in pharmaceutical drug research. With the increasing employment of automation techniques over the last years within Drug Discovery processes, methods like High-Throughput-Screening (HTS) and High-Throughput-Synthesis have become industry standards in pharmaceutical research. Nowadays, it is possible to test more than 20,000 molecules per day for their biological activities in certain disease targets. Also in the area of chemical synthesis, combinatorial chemistry in combination with automation processes, hundreds of molecules per day can be made physically available. Since based on today's chemical knowledge, more than 10¹⁰⁰ molecules could theoretically be synthesized and tested and several hundreds of thousands molecules are commercially available, computer assisted methods have been developed to select subsets of molecules which are actually supposed to be tested based on their predicted potential of biological activity for certain disease targets.

Two categories of computer assisted methods serve the purpose of discovering (selecting and/or prioritizing) molecules from data sets of theoretically available molecules for biological activity testing. The first category comprises diversity or similarity based discovery methods, whereas the second category comprises structure based discovery methods. Among the second category, there are database search techniques, as well as (Q)SAR methods and Docking methods.

Only the (Q)SAR methods and the Docking methods implicitly consider information related to specific targets, either common structural patterns of a series of active molecules ((Q)SAR) or the 3-dimensional structure of a target protein (Docking) and therefore deliver the most specific results. In practice, methods based on (Q)SAR or Docking are applied to smaller data sets (up to 50,000 sets), since they need relatively high computing power. However, although parallel computing techniques can be used to gain speed, still data sets consisting of more than 10⁶ molecules are not predictable with respect to their biological activity in a reasonable time frame.

The term biological activity is hereinafter used to comprise in particular pharmaceutical as well as agrochemical activity with respect to a certain receptor or target.

The search for candidate molecules also comprises the search for lead compounds.

It is therefore an object of the present invention to provide a method of and a system for finding candidate molecules expected to be biologically active, which method and system can be applied on molecule libraries comprising high amounts of data and yields results in a reasonable time.

This object is achieved by the method, the system, and the devices according to the independent claims. Advantageous embodiments are defined in the dependent claims.

Accordingly, one aspect of the invention is a method of identifying biologically active molecules from a set (S) of a predetermined number (N) of different molecules (M1, M2, . . ., MN), said molecules being expected to be biologically active with respect to a predetermined target (T), each said molecule (M1, M2, . . ., MN) of said set (S) being identified by a machine-readable descriptor (X1, X2, . . ., XN), respectively, each said descriptor (X1, . . . , XN) being a vector with n vector elements (x1, . . . , xn), n being a natural number, each vector element (x1, . . . , xn) representing a predetermined molecular property, said method comprising the following steps:

a) selecting said set (S) of molecules as initial set (SE) of evaluation, and a first molecule selection scheme as molecule selection scheme (FS);

b) selecting, according to the selected molecule selection scheme (FS), from said evaluation set (SE) a predetermined first number of molecules as centroid molecules (Mc);

c) grouping each molecule (Mi) of said evaluation set (SE) to the one centroid molecule (Mc) to which the molecule (Mi) has the smallest distance (D), said distance (D) being determined based on a predetermined metrics applied on the descriptor (Xi) of said molecule (Mi) to be grouped and the respective descriptors of said centroid molecules (Mc); all the molecules grouped to one centroid molecule (Mc) forming a cluster (Ci) of molecules of the respective centroid molecule (Mc);

d) for each said cluster (Ci): computing a quality factor (I) according to a predetermined quality criterion, by evaluating the respective affinity values (f) of a second predetermined number of molecules grouped to said cluster (Ci);

e) determining the one cluster (Cb) having the best quality factor (I), and for said determined cluster (Cb): if not already done, searching, among the molecules of said cluster (Ci), the pair of molecules (P1,P2) having the maximum function value f_{D}(P1,P2); marking said pair of molecules (P1,P2); calculating virtual molecule A; searching for and evaluating existing molecule A′ most similar to said molecule A;

f) as long as a predetermined stop criterion (STC) is not reached: selecting each of the clusters (Ci) which satisfies a predetermined split condition (SC) as a new set of evaluation (SE), and repeating steps b) to d) on each said new evaluation sets (SE) separately, whereby a second molecule selection scheme is applied as molecule selection scheme (FS); and then repeating steps e) and f);

g) Outputting the marked molecules.

According to the invention, only a very small amount of molecules within the data set have to be really calculated. This results in a considerable gain of performance. The iterative proceeding allows to study the data base based on customizable quality criteria.

Thus, as examples have shown, active molecules can be identified from data sets by explicitly calculating/measuring just 10% of the molecules within the set of molecules. Thus, large molecule data bases can be exploited compared to standard methods. A further advantage of the invention is that the search for candidate molecules can be performed for several targets in parallel.

By using the method according to the invention, drug lead candidates can be identified without the need of making large molecule sets physically available and testing them. The outputted molecules are suitable for chemical synthesis.

Preferably, said first molecule selection scheme (FS) comprises selecting arbitrarily a predetermined number of molecules, said predetermined number of molecules being substantially smaller than the total number of molecules of said evaluation set (SE).

And preferably, said second molecule selection scheme comprises selecting arbitrarily two molecules of the respective cluster (Cj).

Further preferably, said predetermined second number of molecules of said cluster (Ci) equals two, said molecules being selected by

determining the one molecule (Md1) which has the greatest distance (D) to said centroid molecule (Mc), said distance (D) being computed based on a predetermined metrics;

determining the one molecule (Md2) which has the greatest distance to said molecule (Md1) having the greatest distance (D) to said centroid molecule (Mc), said distance (D) being computed based on said predetermined metrics.

Preferably, the molecular properties represented by said descriptors are at least two of:

molecular weight,

number of rotatable bonds,

number of hydrophobic groups,

number of hydrophilic groups,

number of acid groups,

number of basic groups,

number of neutral groups,

number of zwitter groups,

number of heavy atoms,

number of H-bond donors,

number of H-bond acceptors,

number of 1-2 dipoles,

number of 1-3 dipoles,

number of 1-4 dipoles.

The invention comprises also a computer system having means for performing the identifying method, means for inputting commands to the system, and means for outputting the result of performing the method. Furthermore, the invention comprises data storage means for storing computer software and data for implementing the invention.

The invention and examples thereof are described in detail with reference to the accompanying figures, in which

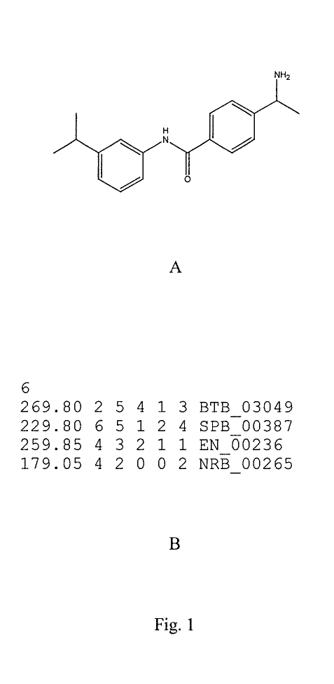

FIG. 1 a 2-D structure of a molecule, and illustrates the type of descriptor used herein,

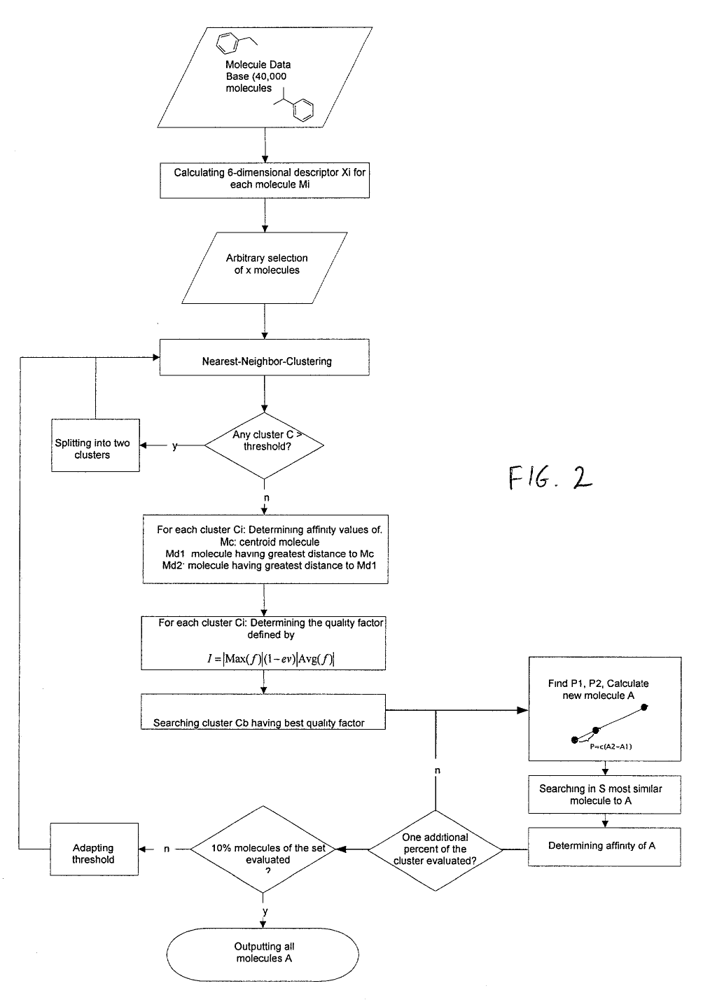

FIG. 2 illustrates the clustering algorithm according to an embodiment of the invention,

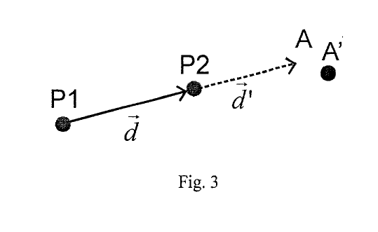

FIG. 3 displays the maximum search in a cluster, and

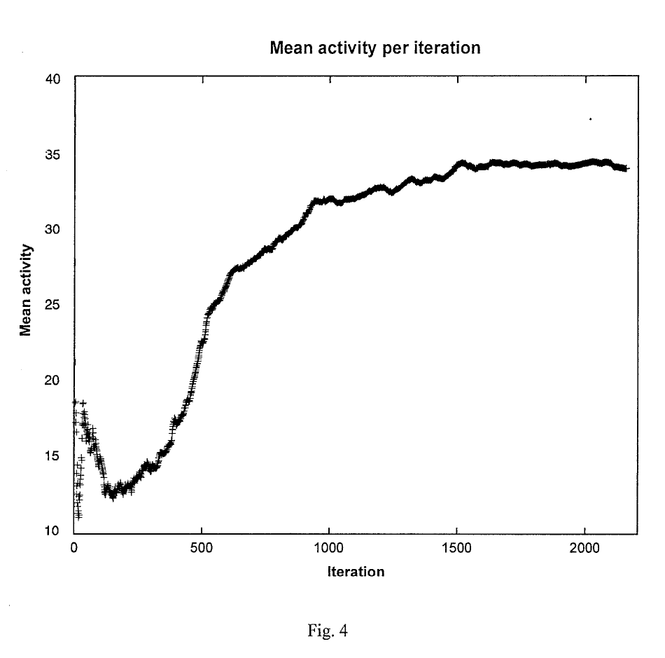

FIG. 4 displays the changes in the mean activity during the calculation.

According to the invention, prior to evaluation of particular molecules, a so-called virtual library S is created, which comprises all possible molecules M. That means that the virtual molecule library contains such molecules which can be purchased or produced with reasonable costs, that are commercially available molecules or molecules which can be produced using combinatorial synthesis approaches. Not be comprised should molecules which are a priori not suitable for drug synthesis, in particular such molecules which contain toxic groups, or which have a molecular weight greater then 500 u or more than 5 donors, or molecules having a log P value of greater than 5. The library is organized as a computer database. The database in this example comprises 40,000 molecules from the World Drug Index. Each of the molecules is represented by 2-D structural data in a machine-readable form. An exemplary 2-D molecule structure is graphically shown in FIG. 1A.

Upon storing the molecules in the library, a descriptor X is assigned to each molecule M of the library, which descriptor X correlates with the biological activity of the respective molecule M. The descriptor X is a vector (x₁, . . . , xₙ) of several molecular properties, each property described by a scalar value xᵢ. This vector X comprises as elements (x₁, . . . Xₙ) the following molecular properties:

molecular weight,

number of rotatable bonds,

number of hydrophobic groups,

number of heavy atoms,

number of H-bond donors,

number of H-bond acceptors.

In order to perform a pre-selection of molecules, it is possible to use values covering economical or technical aspects, such as availability and production costs of molecules.

FIG. 1B displays, as an example, four vectors (denoting four molecules) of the descriptor used in this example. The first line specifies the dimension of the descriptor (6), the second to fifth lines specify the molecules, whereby the last element of each vector contains the ID of the corresponding molecules.

The descriptors X are adapted for further processing the molecule library S in order to find out the best molecule candidates for drug synthesis. In order to allow further processing, the descriptors chosen for the molecules of the database are all of the same dimension.

The most straightforward approach to search those molecules having the highest values of biological activity over the molecule distribution, would consist in directly computing the biological activity of all the molecules of the library. However, such an exhaustive approach would be too much time consuming. Therefore, a faster search has to be performed. According to the invention, this search is performed by applying a clustering algorithm (CA).

FIG. 2 show the steps of an embodiment of the inventive method including the CA.

In the first step, 0.1% of all molecules of the dataset are arbitrarily selected. The selection can be performed by taking random numbers between 1 and the number of molecules in the database, here: 40,000. Another approach is to select the molecules such that the diversity is maximized. However, this leads to higher computation times. Each of these molecules forms a centroid molecule to which the other molecules are grouped. The grouping is performed in such a way that every molecule of the set S is grouped to the one centroid molecule to which it has the smallest distance (“nearest neighbour”), whereby the distance is determined from the respective descriptors of the molecule to be grouped and the centroid molecule. As a measure for the distance between such an descendant and a molecule, the Euclidean distance D of the respective descriptors X, Y is used,

$$D ( X , Y ) = \vert X , Y \vert = \sqrt{{\sum }_{i = 1}^{n} {( {x}_{i} - {y}_{i} )}^{2}} ,$$

wherein xᵢ denotes a vector element of the first descriptor X, and yᵢ denotes a vector element of the second descriptor Y.

Other metrics may be applied, e.g. Cosinus-Coefficient, Tanimoto-Coefficient, Mahalanobis-Distance. This leads to a number of clusters of molecules grouped to the respective centroid molecule.

The intra-cluster similarity should have a chemical meaning, therefore the distance between the molecules of each cluster and their respective cluster centroid should not exceed a predetermined threshold figure.

If the intra-cluster distances are too large, the cluster will be split into two clusters, by setting the outlier molecule as the new centroid and keeping the old centroid of the cluster and grouping the other molecules to the respective closer one of these centroid molecules.

Among the set of clusters thus obtained, the “best” cluster is determined, i.e., the one cluster satisfying best a predetermined quality factor. As quality factor, the respective affinity values of three molecules of a cluster are evaluated. For each cluster, the activity values of the centroid molecule as well as of two other molecules are evaluated. The first molecule is the one molecule, Md1, having the largest distance to the centroid molecule Md0, the distance being computed preferably based on the same metrics as used in the clustering step. The second molecule is the one molecule, Md2, having the largest distance to the first molecule Md1. The affinity values are entered in the following quality factor:

I=|Max(f)|(1−ev)|Avg(f)|,

wherein Max denotes the maximum value of the affinity of a molecule of the cluster Ci to the target T; ev denotes the percentage of evaluated molecules of the cluster Ci;f the affinity of the respective molecule to the target T; Avg notes the average over the evaluated molecules of the cluster Ci.

On the cluster having the best quality factor, the actual maximum search is performed. Hereto, the couple of molecules P1, P2 is searched which has the largest function value of f_{D}(P1,P2) regarding to the target T.

$${f}_{D} = \frac{\vert {f}_{A} ( {P}_{1} ) - {f}_{A} ( {P}_{2} ) \vert}{D ( {P}_{1} , {P}_{2} )} ,$$

f_{A}(P₁: affinity of molecule P₁ regarding to target T;

D(P₁,P₂): distance between P₁ and P₂.

Along the connection line between these two molecules P1, P2, whereas the affinity of P2 is larger than that of P1, the virtual molecule A is calculated according to the following function (see FIG. 3):

$$A = {P}_{2} + {\vec{d}}^{′} with {\vec{d}}^{′} = \vec{d} \cdot \frac{{D}_{max}}{D ( {P}_{1} , {P}_{2} )} \cdot c ,$$

Dₘₐₓ: Maximum intra-cluster distance;

c: scaling factor, typical value 0.3.

Then, the most similar molecule to A existing in the dataset is selected, denoted as A′.

The affinity f may be computed by use of a docking program. For computation of the affinity, reference is made to: B. Kramer, M. Rarey, and T. Lengauer: “Evaluation of the FlexX incremental construction algorithm for protein-ligand docking PROTEINS: Structure, Functions, and Genetics”, Vol. 37, pp. 228-241, 1999, or T. Lengauer and M. Rarey: “Computational Methods for Biomolecular Docking Current Opinion in Structural Biology”, Vol. 6, pp. 402-406, 1996.

In the next iteration, threshold figure for the maximum number of molecules grouped to one cluster is diminished. Accordingly, the all the clusters which exceed the new threshold, are split into two smaller clusters as described above. For each new cluster so formed, the respective quality factor is determined according to the criterion described above. Then the search for the best cluster Cb is made, as described above. For that cluster, the maximum search is performed (if not yet performed in one of the preceding steps); the molecule found is marked “P”.

The process of clustering, searching the best cluster and searching a maximum affinity value in the best cluster is repeated until ten percent of molecules have been evaluated. Then, all the marked “A” molecules are outputted.

The performance of the method according to the invention was evaluated with a 40,000 molecule Set of the World Drug Index. The inhibition of the enzyme scd1 was measured in terms of target-receptor-affinity.

The time needed for the evaluation of the subset was 8020 minutes (2 minutes per molecules, 20 minutes for the cluster algorithm), whereby the CA algorithm was implemented in C++ and was run on a 400 MHz computer system. The data base was based on a Oracle 8.15 RDBMS.

The identified molecules may be tested in suitable biological assays as described for instance by R. Bolger, “High-throughput screening: new frontiers for the 21^{st} century”, published in DDT, Vol. 4, No 6, pp. 251-253, June 1999, or by J. S. Major, “Challenges of high throughput screening against cell surface receptors”, J. of Receptor and Signal Transduction Research, 15(1-4), pp. 595-607, 1995.

FIG. 4 displays the changes in the mean activity during the calculation. One iteration includes finding the cluster with the best quality factor and evaluating 1% of this cluster.

1. A method of identifying biologically active molecules from a set (S) of a predetermined number (N) of different molecules (M1, M2, . . ., MN), said molecules being expected to be biologically active with respect to a predetermined target (T), each said molecule (M1, M2, . . ., MN) of said set (S) being identified by a machine-readable descriptor (X1, X2, ., XN), respectively, each said descriptor (X1, . . . , XN) being a vector with n vector elements (x1, . . . , xn), n being a natural number, each vector element (x1, . . . , xn) representing a predetermined molecular property, said method comprising the following steps:
h) selecting said set (S) of molecules as initial set (SE) of evaluation, and a first molecule selection scheme as molecule selection scheme (FS);
i) selecting, according to the selected molecule selection scheme (FS), from said evaluation set (SE) a predetermined first number of molecules as centroid molecules (Mc);
j) grouping each molecule (Mi) of said evaluation set (SE) to the one centroid molecule (Mc) to which the molecule (Mi) has the smallest distance (D), said distance (D) being determined based on a predetermined metrics applied on the descriptor (Xi) of said molecule (Mi) to be grouped and the respective descriptors of said centroid molecules (Mc); all the molecules grouped to one centroid molecule (Mc) forming a cluster (Ci) of molecules of the respective centroid molecule (Mc);
k) for each said cluster (Ci): computing a quality factor (I) according to a predetermined quality criterion, by evaluating the respective affinity values (f) of a second predetermined number of molecules grouped to said cluster (Ci);
l) determining the one cluster (Cb) having the best quality factor (I), and for said determined cluster (Cb): if not already done, searching, among the molecules of said cluster (Ci), the pair of molecules (P1,P2) having the maximum function value f_{D}(P1,P2); marking said pair of molecules (P1,P2); calculating virtual molecule A; searching for and evaluating existing molecule A′ most similar to said molecule A;
m) as long as a predetermined stop criterion (STC) is not reached: selecting each of the clusters (Ci) which satisfies a predetermined split condition (SC) as a new set of evaluation (SE), and repeating steps b) to d) on each said new evaluation sets (SE) separately, whereby a second molecule selection scheme is applied as molecule selection scheme (FS); and then repeating steps e) and f);
n) Outputting the marked molecules.

2. The method according to claim 1, wherein said first molecule selection scheme (FS) comprises selecting arbitrarily a predetermined number of molecules, said predetermined number of molecules being substantially smaller than the total number of molecules of said evaluation set (SE).

3. The method according to claim 1, wherein said second molecule selection scheme comprises selecting arbitrarily two molecules of the respective cluster (Cj).

4. The method according to claim 1, wherein said predetermined second number of molecules of said cluster (Ci) equals two, said molecules being selected by
determining the one molecule (Md1) which has the greatest distance (D) to said centroid molecule (Mc), said distance (D) being computed based on a predetermined metrics;
determining the one molecule (Md2) which has the greatest distance to said molecule (Md1) having the greatest distance (D) to said centroid molecule (Mc), said distance (D) being computed based on said predetermined metrics.

5. The method according to claim 4, wherein said quality factor (I) is defined by:
I=|Max(f)|(1−ev)|Avg(f)|,
wherein
Max denotes the maximum value of the affinity of a molecule of said cluster (Ci) to said target (T);
ev denotes the percentage of evaluated molecules of the cluster (Ci);
f the affinity of the respective molecule to said target (T);
Avg denotes the average over the evaluated molecules of the cluster (Ci).

6. The method according to claim 1, wherein said molecule having the maximum affinity value in step f) is determined by:
searching the couple of molecules (P1, P2) having the largest function value f_{D}(P1,P2);
searching, along the distance vector between the couple of molecules (P1, P2) found, the one point (A) having the maximum affinity; preferably according to the following function:
A=P₂+\vec{d}′,
with
$${\vec{d}}^{′} = \vec{d} \cdot \frac{{D}_{max}}{D ( {P}_{1} , {P}_{2} )} \cdot c ,$$
wherein the affinity value of P1 is larger than the affinity value of P2,
f(P₁)>P₂;
searching the one molecule A′ of said set (S) of molecules having the most similar descriptor to said determined point (A).

7. The method according to claim 1, wherein said metrics is defined by:
$${D}_{xy} = \sqrt{{\sum }_{i = 1}^{n} {( {x}_{i} - {y}_{i} )}^{2}}$$
with
xᵢ: vector element of said first descriptor,
yᵢ: vector element of said second descriptor,
n: number of vector elements of said first and second descriptor, respectively.

8. The method according to claim 1, wherein said stop criterion is defined by reaching a predetermined number of repetitions of steps b) to e).

9. The method according to claim 1, wherein said stop criterion is defined by reaching a predetermined percentage of molecules of said set (S) having been evaluated so far.

10. The method according to claim 1, wherein in step d), each cluster is split which satisfies said predetermined split condition.

11. The method according to claim 1, wherein said split condition is given by a predetermined number of molecules of the respective cluster (Ci).

12. The method according to claim 1, comprising a step of visualizing the outputted molecules.

13. The method according to claim 1, wherein said set of molecules is held in a computerized database.

14. The method according to claim 1, comprising a step of visualizing the resulting 3-D surfaces.

15. The method according to claim 1, wherein said selected candidate molecules are suitable for chemical synthesis.

16. The method according to claim 1, whereby the molecular properties represented by said descriptors are at least two of:
molecular weight,
number of rotatable bonds,
number of hydrophobic groups,
number of hydrophilic groups,
number of acid groups,
number of basic groups,
number of neutral groups,
number of zwitter groups,
number of heavy atoms,
number of H-bond donors,
number of H-bond acceptors,
number of 1-2 dipoles,
number of 1-3 dipoles,
number of 1-4 dipoles.

17. The method according to claim 1, whereby the molecular properties represented by said descriptors are:
molecular weight,
number of rotatable bonds,
number of hydrophobic groups,
number of heavy atoms,
number of H-bond donors,
number of H-bond acceptors.

18. The method according to claim 1, whereby the molecular properties represented by said descriptors are at least two of:
molecular weight,
number of rotatable bonds,
number of hydrophobic groups,
number of heavy atoms,
number of H-bond donors, number of H-bond acceptors.

19. A computer system comprising means for performing the method according to claim 1.

20. The computer system according to the preceding claim comprising means for communicating with a database comprising said set of molecules.

21. A data storage means storing a program for performing the method according to claim 1.

22. A data storage means storing a database comprising the set of molecules for use with the method according to claim 1.

23. A program for storing a database comprising the set of molecules for use with the method according to claim 1.

24. A database to be used with the method according to claim 1.

25. Method of producing molecules determined by the method according to claim 1.

26. Method according to claim 25, further comprising a final step of testing said found candidate molecules in a suitable biological assay.
